(19) United States
(12) Patent Application Publication
(10) Pub. No.: US 2020/0160739 A1
(43) Pub. Date: May 21, 2020

(54) DIGITAL WEB-BASED EDUCATION PLATFORM FOR DELIVERING TARGETED AND INDIVIDUALIZED TRAINING ON MEDICAL CONDITION MANAGEMENT TO USERS

(71) Applicant: Becton, Dickinson and Company, Franklin Lakes, NJ (US)

(72) Inventors: Michael SMITH, Ivybridge, Devon (GB); Carmel MCDERMOTT, Reaumont (FR); Kenneth STRAUSS, Erembodegem (BE); Timothy VAN DER SCHRAELEN, Nyon (CH); Matthew ZUSCHLAG, Randolph, NJ (US); Marcus SILVA, Fairfield, NJ (US); Jade CANTOR, Englewood, NJ (US); Claire Lotito LEVINE, Wayne, NJ (US); Mojtaba KASHEF, Boxford, MA (US)

(73) Assignee: Becton, Dickinson and Company, Franklin Lakes, NJ (US)

(21) Appl. No.: 16/473,392

(22) PCT Filed: Dec. 27, 2017
(86) PCT No.: PCT/US2017/068486

§ 371 Date: Jun. 25, 2019

Related U.S. Application Data
(60) Provisional application No. 62/440,163, filed on Dec. 29, 2016.

(51) Int. Cl. G09B 5/02; G09B 19/00; G16H 10/60

(52) U.S. Cl. CPC G09B 5/02; G16H 10/60; G09B 19/00

(57) ABSTRACT

A digital web-based education platform is provided for delivering targeted and individual training (e.g., to patients on injection technique to help with medical condition management). An associated website is accessible by users via computers and mobile devices. The platform has several short learning modules (e.g., animations, videos that can include knowledge assessment) on various topics. Healthcare providers (HCPs) can prescribe or recommend which topics each patient should follow and prescribes a selected product having a code. Patients access the learning modules using the website by entering the code found in the box or packing for a product, or provided by an HCP, and review learning modules and topics of their choice or recommended by an HCP using a product code and the website URL. The platform collects patient usage data and metrics that can be used to provide incentives to patients,

### BACKGROUND OF THE INVENTION

### Field of the Invention

The present invention relates to systems, methods and apparatuses for providing tailored information to users via a digital, web-based education platform. The present invention also relates to optimizing patient education and adherence to a medical condition treatment regimen by relating a prescribed product to targeted, individualized access to platform lesson modules and topics selected by or for a particular patient based on that patient's profile and treatment regimen.

### Description of Related Art

People with diabetes often have a poor understanding of their condition and its management and need to be encouraged to work with their healthcare professionals or providers (HCPs) to seek information that could help them manage their condition and achieve better long term quality of life. The ‘Good Practice Forum’ (http://www.efgcp.eu/) highlighted the need for improved information provision for diabetes care and identified a need to help people with diabetes piece together available information in a manner that best helps them achieve their diabetes management goals using the most effective practices.

Unfortunately, many HCPs today have limited professional development opportunities in regard to patient information giving. They often also have a lack of time and poor tools and resources for teaching patients. Thus, they are often faced with poor patient adherence to treatment regimens. In addition, HCPs are often not aware of the best practices with regard to injection technique.

Injection technique, however, has an important role in achieving and maintaining optimal glycemic control. In other words, patients need both treatment compliance (e.g., successfully adhering to prescribed regimen for insulin dosing such as type of insulin, amount, and dosing schedule), and use compliance (e.g., adhering to a proper injection practice). It is increasingly clear that optimal injection technique is a fundamental objective for people with diabetes who use insulin, because improper technique can lead to injections into the muscle, or development of lipohypertrophy in certain body areas from inadequate injection site rotation, both of which negatively impact insulin absorption and therefore glycemic control.

When patients are prescribed self-injection as part of their disease management regimen, they typically receive patient education in the form of in person practical training on injection from their healthcare provider (HCP). Indeed, a significant amount of the time HCPs spend with their diabetes patients is devoted to self-injection training and follow-up counseling on the degree of glycemic control attained from self-injecting.

Self-injection remains a major source of trivial mistakes by patients with possible severe consequences, and requires continuous education. Often HCPs find that patients are overwhelmed by the information received during their patient education trainings. In other words, patients may seem to understand proper self-injection technique at first during the training, and then later forget what they need to do to properly self-inject. Alternatively, patients may have understood the self-injection procedure taught during a patient education training, but still elected to not adopt this procedure. Regardless of whether patients do not understand what they have been told, or forget what they have been told, their adherence to therapy is negatively impacted. Poor adherence leads to poor health outcomes, complications and glycemic crisis. Patients usually have access to only general information post diagnosis, and they receive little or no self-injection training post diagnosis. They are instead likely to follow information from untrained experts such as family members and friends.

### SUMMARY OF THE INVENTION

The above and other problems are overcome, and additional advantages are realized, by illustrative embodiments of the present invention.

It is an aspect of illustrative embodiments of the present invention to provide a system, apparatuses and methods for accessing tailored information from a web-based education platform by storing a plurality of indexed learning modules in a memory storage device; storing a plurality of codes in a memory storage device; registering a user with the platform; receiving a code entered by the user into a user portal to the platform; determining if the entered code is one of the plurality of codes; permitting the user access into the platform and to the indexed education learning modules when the entered code is determined to be from among the plurality of codes; and generating an output of a selected subset of the indexed education learning modules based on one of user selection of the subset of the indexed education learning modules, and selection of the subset by a provider who prescribes training to the user. For example, the education learning modules can be any of video, multimedia, and print materials. By way of another example, the code can be obtained from an injection product or from a healthcare provider (HCP). Also, for example, registering comprises setting a user profile. Further, a user can grant an HCP access to the user profile.

In accordance with another aspect of illustrative embodiments of the present invention, the learning modules can be divided by different lessons relating to different aspects or stages of medical injection, and each lesson has plural topics, and a healthcare provider (HCP) selects the subset based on patient data related to medical condition management. The platform can be configured to generate user metrics relating to a plurality of criteria selected from the group consisting of login frequency, lessons accessed and completed, topics accessed and completed, duration and timing of lesson or topic accesses, dwell times and mouse activity during lesson or topic accesses, and the HCP can prescribe different ones of the lessons and topics based on the user metrics.

In accordance with another aspect of illustrative embodiments of the present invention, the system, apparatuses and methods are configured to allow accessing tailored information from a web-based education platform by activating a code after it is entered into the platform for a selected period of time; providing the user with unlimited access to the indexed education learning modules during the selected period of time; expiring the code after the selected period of time has elapsed; and denying the user access to the indexed education learning modules after the code has expired. For example, a user can enter a second code into the platform portal to gain permission to access to the indexed education learning modules again for a selected period of time until the second code expires.

In accordance with another aspect of illustrative embodiments of the present invention, the learning modules are divided by different lessons relating to different aspects or stages of medical injection, and each lesson has plural topics, and the system, apparatuses and methods are configured to generate user metrics relating to a plurality of criteria selected from the group consisting of login frequency, lessons accessed and completed, topics accessed and completed, duration and timing of lesson or topic accesses, dwell times and mouse activity during lesson or topic accesses. An HCP can review the user metrics generated during the respective selected time periods related to the code and second code and prescribe different ones of the lessons and topics based on the user metrics. Further, for example, the HCP can prescribe different ones of the lessons and topics based on patient data related to medical condition management.

In accordance with another aspect of illustrative embodiments of the present invention, the system, apparatuses and methods are configured to allow accessing tailored information from a web-based education platform by generating user metrics relating to a plurality of criteria selected from the group consisting of login frequency, lessons accessed and completed, topics accessed and completed, duration and timing of lesson or topic accesses, dwell times and mouse activity during lesson or topic accesses; storing patient data related to medical condition management; providing payers access to the user metrics and patient data; and rewarding at least one of a patient and healthcare provider with incentives to encourage the patient consumption of the indexed education learning modules.

In accordance with another aspect of illustrative embodiments of the present invention, an energy harvesting display device displays content regarding the platform; wirelessly couples with a mobile phone and transmits stored content to the mobile phone; and plays the stored content on the mobile phone. For example, the mobile phone can be operated to navigate to a website of the platform; generate a screen with a field in which to enter the code; and play back selected ones of the subset of the indexed education learning modules via the mobile phone only after the code is entered. Further, the example, the energy harvesting display device can be placed on a product, wherein the product has a unique code stored in the energy harvesting display device; and can display the code for the user to manually enter into the field, or automatically fill the field in with the code via the wireless link between the mobile phone and the energy harvesting display device.

It is an aspect of illustrative embodiments of the present invention to provide a web-based education platform connected to least one user device via a communication network, the user device having a user output device and a user input interface, platform comprising: a memory storage device comprising instructions; a plurality of indexed learning modules stored in the memory storage device; a plurality of codes stored in the memory storage device; and a graphical user interface (GUI) module configured to generate screens for display on the user output device; at least one processor in communication with the memory storage device and the GUI module. The processor executes the instructions to: restrict access to the learning modules until a code from among the plurality of codes has been received and activated; receive a first signal indicative of a user registration of the user device with the platform and, in response to the first signal, generate a user profile for that user comprising at least user identification and storing the user profile in the database; receive a second signal indicative of a code inputted by the user via the user input interface; and in response to the second signal, determine if the code inputted by the user is from among the plurality of codes and is not activated for another user. When the code inputted by the user is determined to be from among the plurality of codes and not activated for another user, the processor executes instructions to generate and store information in the memory storage device indicative of association of the code with the user, and configure the memory storage device to prevent the association of that code with a different user; activate the code for that user for a selected time period; transmit a third signal indicative of a selected subset of the indexed learning modules for display to the user on a user output device, the selected subset of the indexed learning modules based on one of user selection of the subset of the indexed learning modules and selection of the subset by a provider who prescribes training to the user; receive a fourth signal indicative of at least one of the selected subset of the indexed learning modules being identified by the user for display on the user output device and, in response to the fourth signal, output the identified learning module to the user device; and, in response to expiration of the time period, deactivate the code, and configure the memory storage device to prevent the output of any of the indexed learning modules to the user device until another one of the plurality of codes is inputted by the user and determined to not be activated.

In accordance with another aspect of illustrative embodiments of the present invention, the learning modules are divided by different lessons, and each lesson has plural topics. The processor executes the instructions to obtain user metrics relating to a plurality of criteria selected from the group consisting of login frequency of the user device, lessons accessed and completed via the user device, topics accessed and completed via the user device, duration and timing of lesson or topic accesses by the user device, dwell times and user device mouse activity during lesson or topic accesses.

In accordance with another aspect of illustrative embodiments of the present invention, the processor executes the instructions to store patient data related to medical condition management in the memory storage device and to provide at least one of a healthcare provider device and a medical claims payer device with access to the user metrics and patient data via the platform.

In accordance with another aspect of illustrative embodiments of the present invention, the medical claims payer device can provide the platform with incentives to users for completing the indexed learning modules and reward criteria for bestowing the incentives to the users. The processor executes the instructions to analyze the user metrics and provide the incentives to the users when the reward criteria are met.

In accordance with another aspect of illustrative embodiments of the present invention, a healthcare provider device is configured to receive the user metrics with respect to each of a plurality of user devices, to prescribe access to at least a selected one of the indexed learning module for respective ones of the plurality of user devices based on their corresponding user metrics, and to generate and provide prescriptions for the plurality of user devices to the platform, the processor executes the instructions to correlate the prescriptions for the plurality of user devices with registration information for respective user devices to output the prescribed indexed learning modules.

In accordance with another aspect of illustrative embodiments of the present invention, the learning modules are divided by different lessons relating to different aspects or stages of medical injection selected from the group consisting of preparing injection device for delivery, selecting injection sites, selecting needle length, detecting lipohypertrophy, caring for injection sites, medication types, injection technique, and storage and disposal of medical injection supplies.

In accordance with another aspect of illustrative embodiments of the present invention, the processor executes the instructions to activate the access code for a selected period of time after it is entered into the platform portal via the user interface, provide the user device with unlimited access to the indexed learning modules during the selected period of time, retire the access code after the selected period of time has elapsed, and deny the user device access to the indexed learning modules after the access code has expired.

In accordance with another aspect of illustrative embodiments of the present invention, a second product has a second access code, and the processor executes the instructions receive the second access code entered via the user input interface, and provide the user device with access to the indexed learning modules again for a selected period of time until the platform retires the second access code.

Additional and/or other aspects and advantages of the present invention will be set forth in the description that follows, or will be apparent from the description, or may be learned by practice of the invention. The present invention may comprise apparatuses and methods for operating same having one or more of the above aspects, and/or one or more of the features and combinations thereof. The present invention may comprise one or more of the features and/or combinations of the above aspects as recited, for example, in the attached claims.

### BRIEF DESCRIPTION OF THE DRAWINGS

The above and/or other aspects and advantages of embodiments of the invention will be more readily appreciated from the following detailed description, taken in conjunction with the accompanying drawings, of which:

FIG. 1 is a diagram of a system comprising a web-based education platform in accordance with an embodiment of the present invention;

FIG. 2 is a diagram of screens for a graphical user interface to the web-based education platform in accordance with an embodiment of the present invention;

FIGS. 3A and 3B are respective sides or pages of a product packaging insert for use in conjunction with the web-based education platform in accordance with an embodiment of the present invention;

FIG. 4 depicts code indicia printed on product packaging for use in conjunction with the web-based education platform in accordance with an embodiment of the present invention;

FIG. 5 depicts a leaflet with code indicia for use in conjunction with the web-based education platform in accordance with an embodiment of the present invention;

FIGS. 6A, 6B and 6C depict a display device and mobile phone with mobile phone app for use in conjunction with the web-based education platform in accordance with an embodiment of the present invention;

FIG. 7 is a flow chart depicting operations for using a code to gain access to selected topic(s) in selected lesson(s) in the web-based education platform in accordance with an embodiment of the present invention;

FIG. 8 is a flow chart depicting operations for an HCP using the web-based education platform to prescribe selected topic(s) in selected lesson(s) to users in accordance with an embodiment of the present invention; and

FIGS. 9, 10, 11, 12, 13, 14, 15, 16, 17, 18, 19, 20 and 21 are screen shots of a graphical user interface of the web-based education platform in accordance with embodiments of the present invention.

Throughout the drawing figures, like reference numbers will be understood to refer to like elements, features and structures.

### DETAILED DESCRIPTION OF ILLUSTRATIVE EMBODIMENTS

A need exists for giving tailored information that meets the needs of individual patients in order to attain optimal therapy. Providing tailored information reduces risk of overwhelming the patient with too much information at once and allows them to better assimilate the targeted information.

A need also exists for a patient education program that allows patients repeated access to the tailored information on the patient's own time and schedule. Patients would then be able to review training information when they feel they have forgotten part of their patient education training. Further, repeated viewing of certain topics would then be possible, which may convince them not to ignore a preferred injection technique.

While accessing the internet can be potentially a good source of information about diabetes, it is not necessarily a good source for information about self-injection. For example, there are few web-based sources dedicated to self-injection. Older diabetes patients may not have the means to access the internet. Further, many patients are unable to discern if the internet information they receive is reliable and has HCP approval or official certifications for its quality and accuracy. Accordingly, a need also exists for a web-based source of prescribed or pre-approved patient education information that is curated, easily modularized and ultimately tailorable to a specific patient with a specific patient profile and treatment regimen.

In addition to the above-described issues with patient education among patients and HCPs, payers are under increasing pressure from both a cost and health outcome perspective with respect to chronic disease management such as diabetes patient management. Glycemic crisis and long term complications are adding significant cost and burden due to the need for emergency care pressures, bed blocking, unplanned admissions and ambulance call outs. Payers are also faced with staff shortages, whereby there is little time for training, given patient volume or throughput.

A need therefore exists for an optimized patient education platform that optimizes patient treatment regimen adherence which leads to improved patient outcomes and reduced healthcare costs. A need also exists for a patient education platform that allows product manufacturers and suppliers to give incentives to HCPs, patients and/or payers, and allows payers to give incentives to HCPs and/or patients, to encourage proper use of a given product and patient adherence to a prescribed injection technique and to encourage patient motivation to seek further training on their disease management regimen.

The above and other problems are overcome, and additional advantages are realized, by illustrative embodiments of the present invention. Reference will now be made in detail to embodiments of the present invention, which are illustrated in the accompanying drawings. The embodiments described herein exemplify, but do not limit, the present invention by referring to the drawings. It will be understood by one skilled in the art that this disclosure is not limited in its application to the details of construction and the arrangement of components set forth in the following description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

### Overview

In accordance with an illustrative embodiment of the present invention, a web-based education platform is provided which has a responsive website available on users' personal computer (PC), tablets and mobile phones. The platform is configured for use by patients (e.g., patients who inject insulin), patients' families (e.g., family members and caregivers), and healthcare professionals (e.g., HCPs involved in the management of people with diabetes who inject insulin). The web-based education platform provides patients, caregivers and family members with a series of learning modules on medical condition management such as diabetes treatment.

The look and feel of the website and related mobile phone app (e.g., a personalized learning portal) is simple to use, and can be configured to reflect a particular medical product brand. The learning modules are a series of multi-lingual, patient-centered education programs or lessons with various topics that can be selected for and thereby tailored to the individual patient (e.g., by user selection, or selection by the patient's clinician or HCP based on the patient profile and prescribed medical treatment regimen). Access to the learning modules can be gained upon entry of a code. The web-based education platform uses the codes and the registrations of users to allow for tracking of users' completion of whichever ones of the learning modules that were selected by the users, or selected for the users by their HCPs. Such tracking permits users to get incentives or rewards for their training efforts using the platform and/or for their improved medical outcomes, or for HCPs or payers to correlate patients' medical outcomes with their training efforts using the platform in order to receive or give awards for patients' improved adherence to a prescribed regimen and/or improved medical outcomes.

The codes can be provided, for example, in or on the boxes of medical supplies or other products for the prescribed treatment regimen (e.g., codes are provided on boxes of pen needles). The website and mobile app are configured to be easily accessible, simple to use, and to provide an optimal user experience. The web-based education platform is configured to allow patients using, for example, the pen needles, and optionally healthcare professionals, to easily access the information they need at the time they need it most. The web-based education platform is described herein with learning modules created to educate diabetes patients who inject insulin by way of an example. It is to be understood, however, that the web-based education platform can be provided with learning modules directed to different topics, medical or non-medical, and configured for access by various types of users besides patients, caregivers and HCPs (e.g., people in various industries or employment fields where training in conjunction with particular products' use facilitates compliance and further training as needed) and for use in conjunction with different types of products.

### Digital Education System

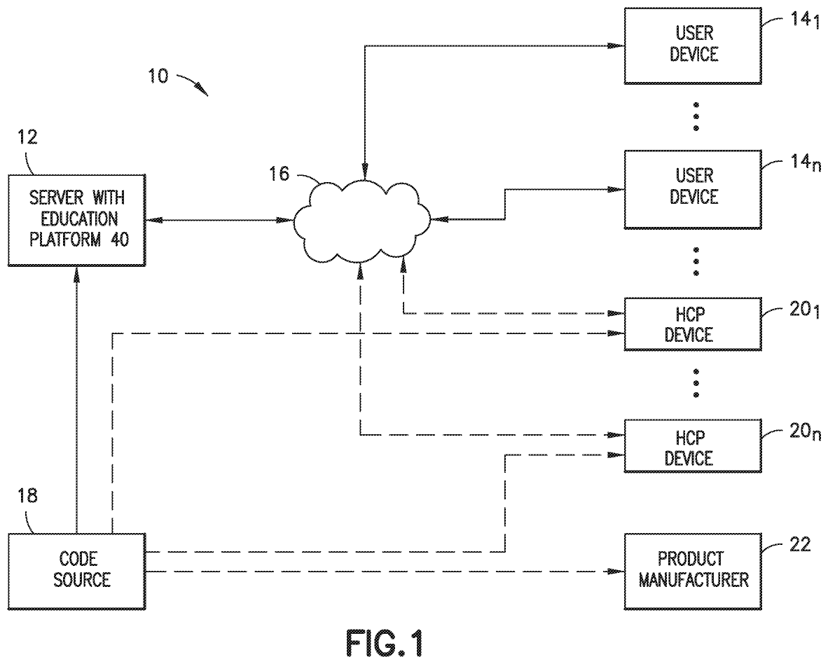

With reference to FIG. 1, an education system 10 comprises a server 12 with web-based education platform 40, which includes a database(s) and other backend infrastructure described below in connection with FIG. 23. Different types of users can access the platform 40 such as patients and their family members or caregivers via user devices 14₁ through 14ₙ, which can be PCs, tablets and mobile phones. Further, other users can optionally be HCPs, or payers, or other stakeholders interested in patients' treatment, medical outcomes and medical costs , who access the web-based education platform 40 via their devices 20₁ through 20ₙ. Their devices 20₁ through 20ₙ are also PCs, tablets and mobile phones, for example.

With continued reference to FIG. 1, user devices 14 access the platform 40, for example, via the internet and/or cellular service as indicated generally by a network 16. HCPs or other stakeholder devices 20₁ through 20ₙ can optionally access the platform 40 via the internet and/or cellular service indicated generally at 16. A code source 18 provides codes for accessing learning modules on the platform 12 and provides these codes to the platform 40 and optionally to a product manufacturer 22 or other supplier.

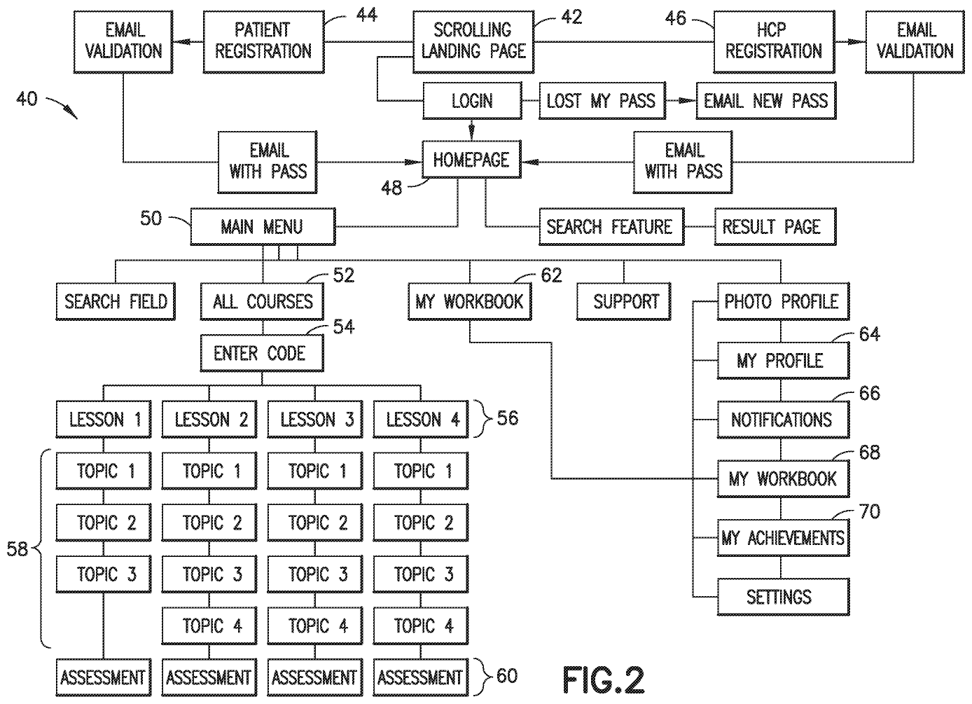

With reference to FIG. 2, the web-based education platform 40 provides a graphical user interface (GUI) (e.g., via a GUI 166 portion of a user interface 164 as shown in FIG. 23) in the form of web pages for viewing on PCs, tablets and mobile phones, for example. As described in more detail below, the web pages guide users (e.g., patients, caregivers and family members, and optionally HCPs and payers) to register with the platform 40, to enter product codes if the user is a patient, and to navigate a customizable set of learning modules on an educational topic such as diabetes (e.g., healthy eating, diabetes medicine, injection techniques, and so on). The learning modules are illustrated as a plurality of lessons 56 which can have one or more topics 58 as shown in FIG. 2. It is understood that the content of the learning modules 56 can have a different organization or structure than lessons with different topics such as, for example, a plurality of lessons or content modules 56 that may or may not have related sub-components such as topics. Further, the learning modules 56 can be organized in a manner that does or does not require that the respective learning modules be viewed in any particular order. In other words, each learning module 56 can be self-contained, or can be related to other learning modules.

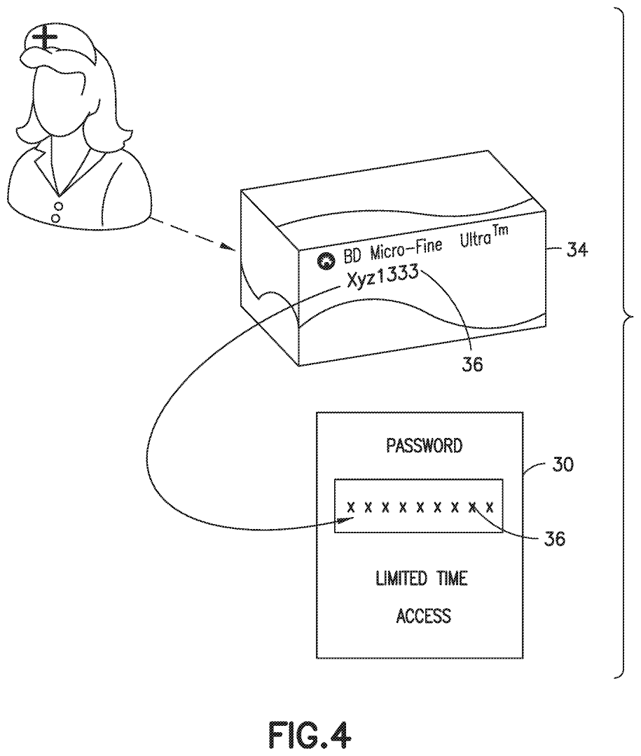

The targeted and individualized set of learning modules 56 is selected by the patient as needed, or selected in response to prescribed training (e.g., the modules or lessons 56 and topics 58 are selected or recommended by the patient's HCP based on the HCP's assessment of the patient's medical condition management and training needs). The platform 40 can provide controlled access to the set of modules through using codes and/or identifiers (IDs) such as user IDs. For example, diabetes management products such as pen needle boxes can be given codes (e.g., code 40 in FIG. 4) that patients or family members and caregivers must enter when they login or register to the platform 40 to view the learning modules.

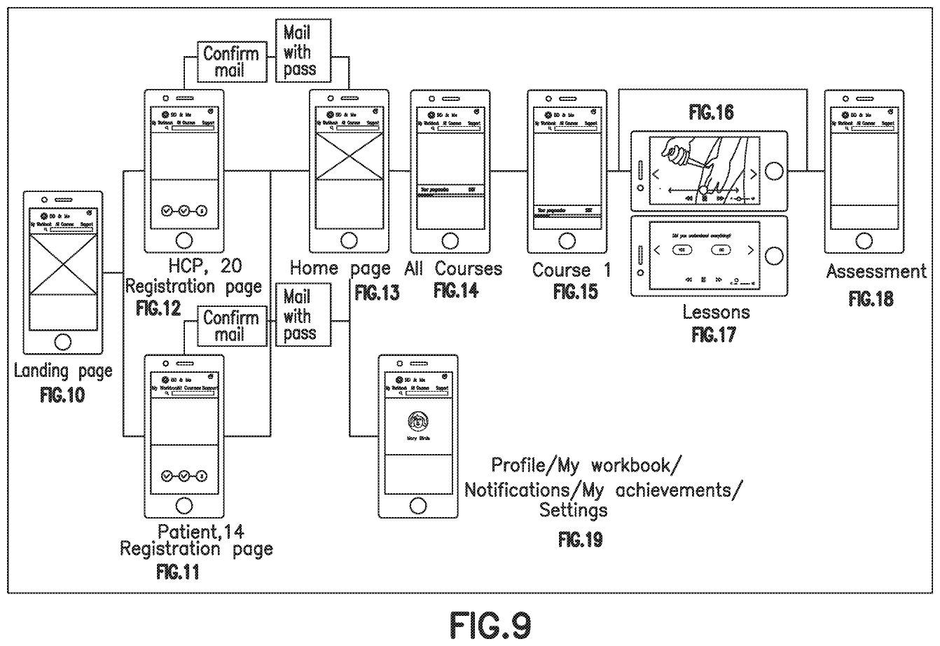

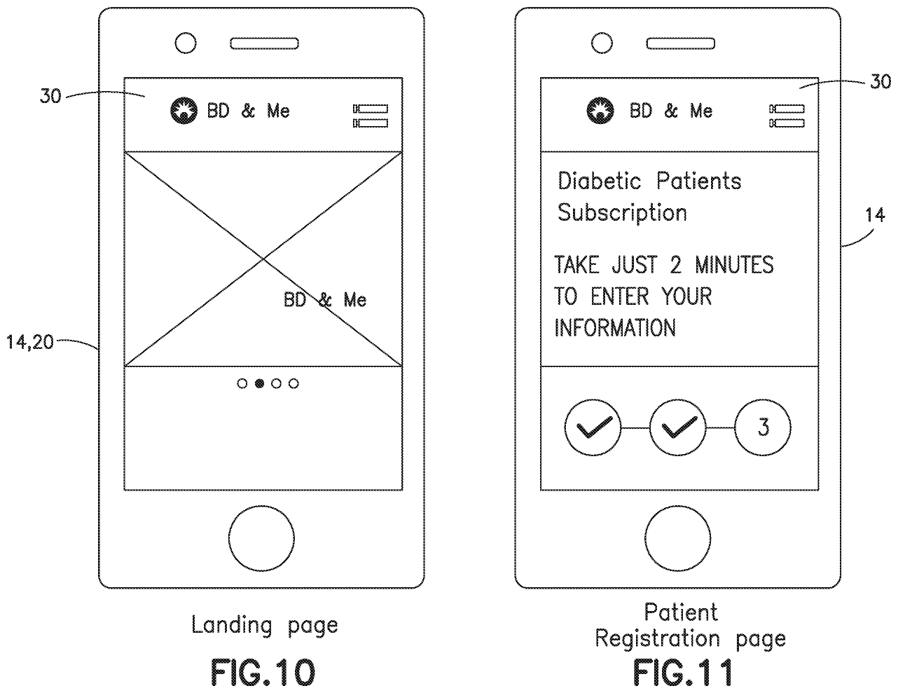

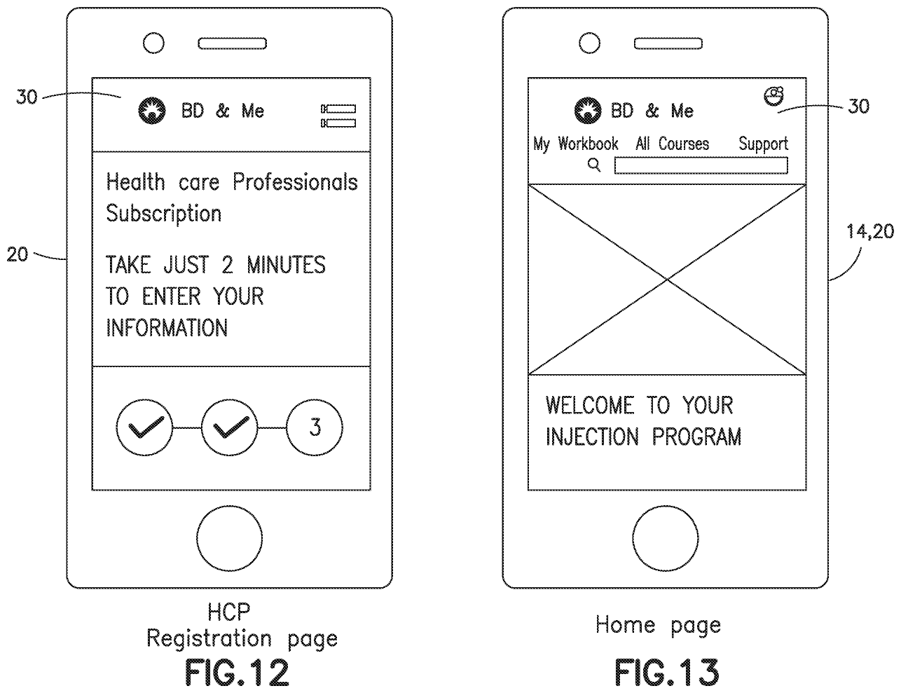

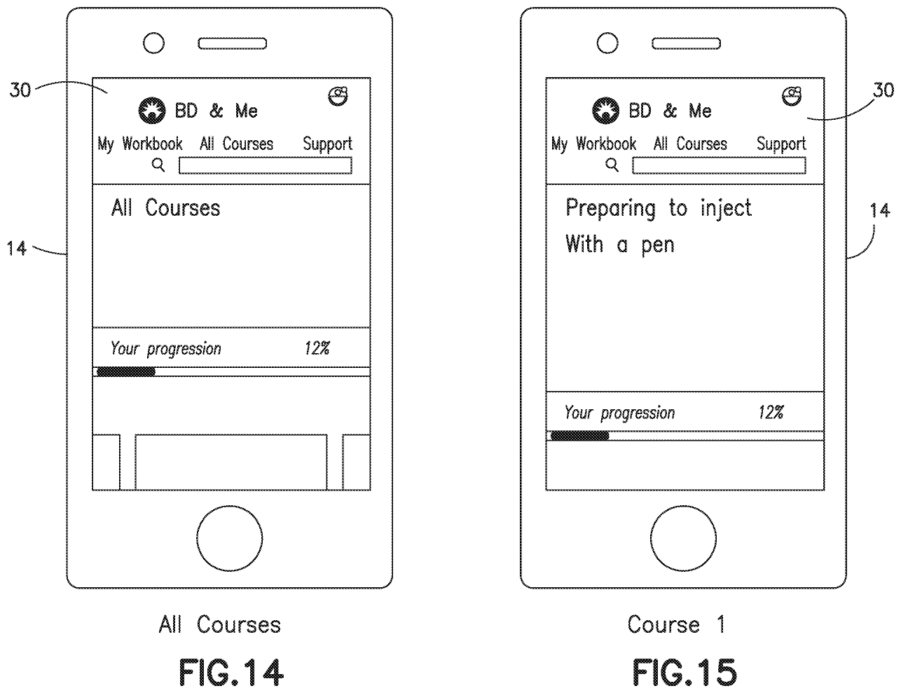

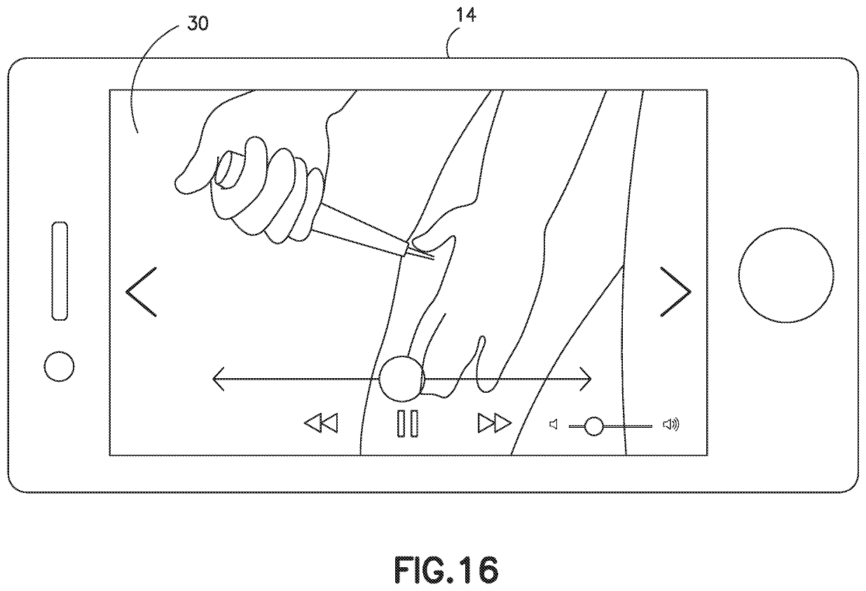

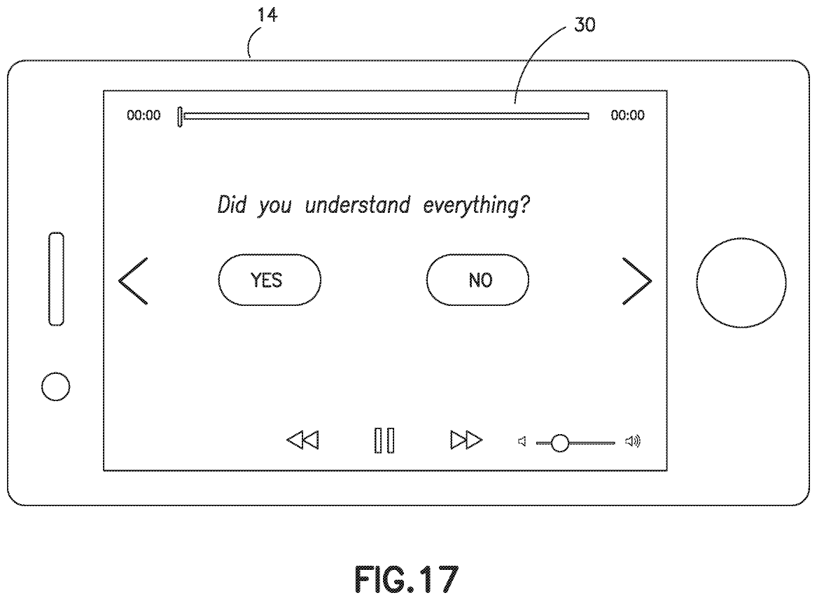

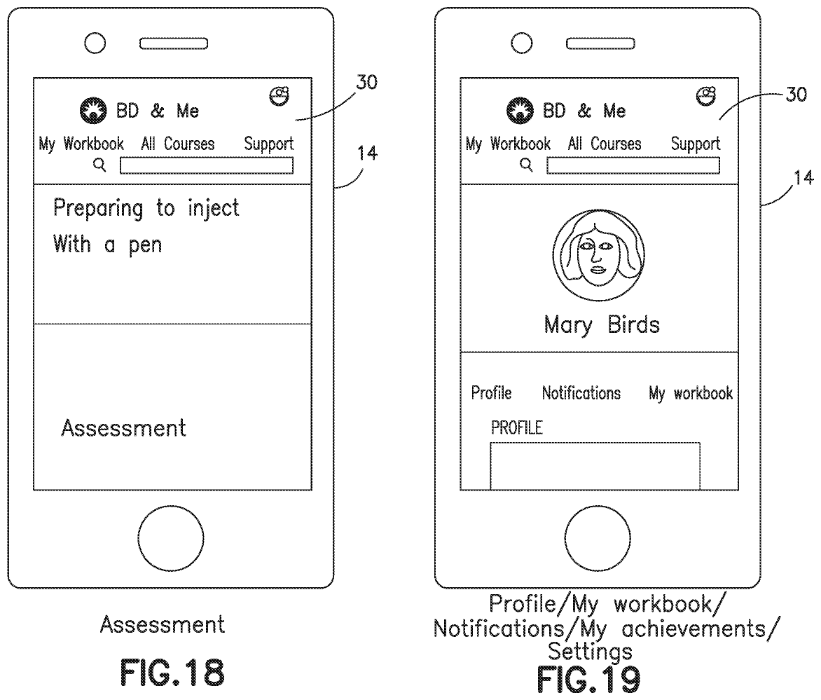

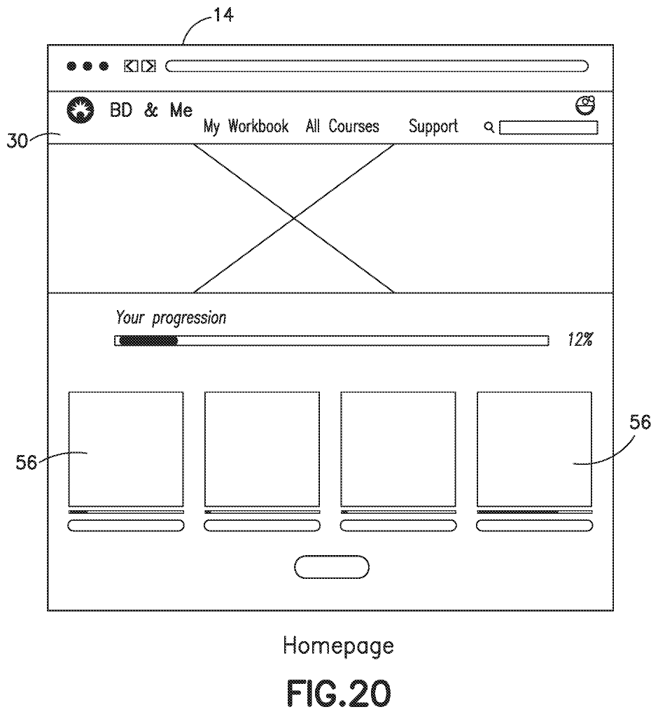

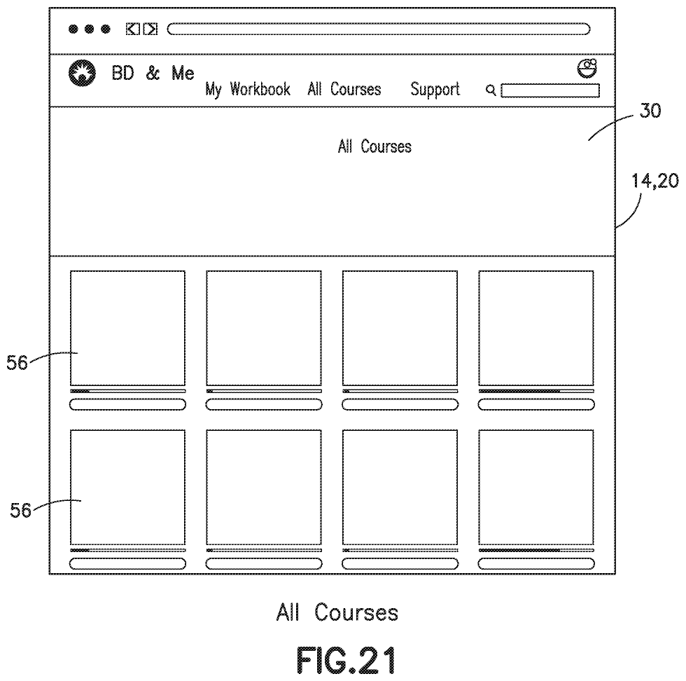

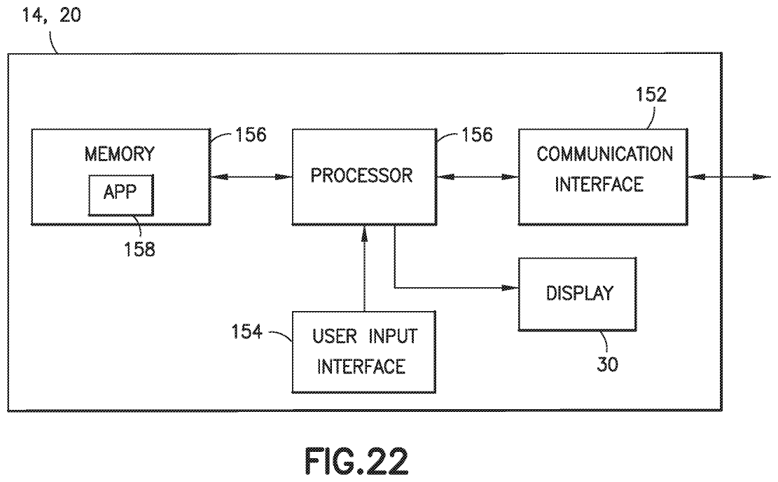

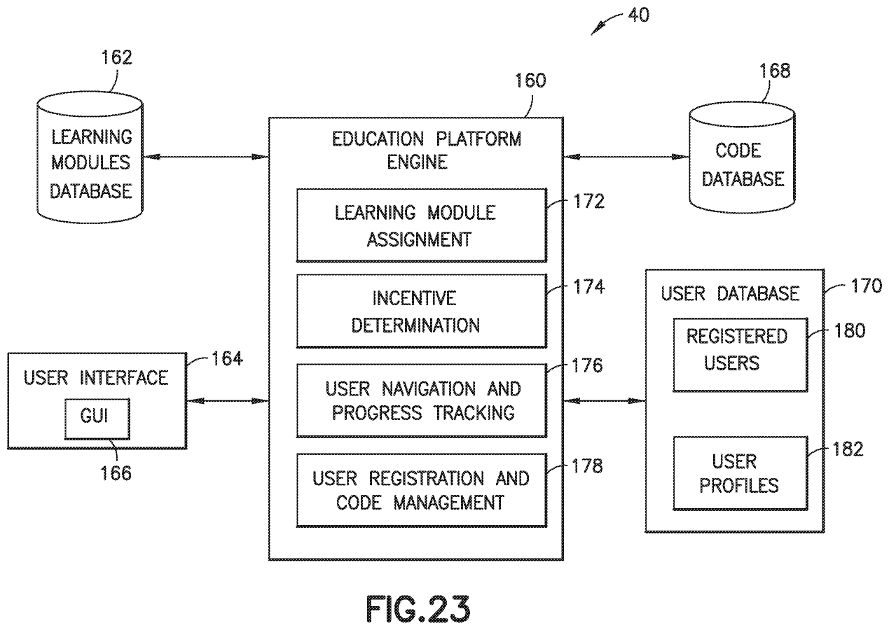

Examples of GUI screens are provided in FIGS. 9 through 21. FIG. 9 is an illustrative screen map for a personalized learning portal app for a device 14 or 20 that is a mobile phone or other device. The respective web pages in the screen map of FIG. 9 are illustrated in more detail in FIGS. 10 through 19. For example, when a device 14, 20 navigates to the landing page (FIG. 10, 42 in FIG. 2) using the URL for the platform 40, the user can identify herself as a patient or HCP and then navigate to the patient registration page or screen portion (FIG. 11, 44 in FIG. 2) or the HCP registration page or screen portion (FIG. 12, 46 in FIG. 2). Either way, a user device 14, 20 navigates to a home page (FIG. 13, 48 in FIG. 2) upon completion of registration. The home page can optionally list learning modules 56 prescribed by the user's HCP, or modules previously selected by the user, as well as provide a progress bar of completion of various learning modules as illustrated in the home page depicted in FIG. 20. The HCP can see, for example, the progress for one of his registered patients. The user can select “All courses” from the home page 48 and see a menu of all of the available learning modules 56 as shown in FIGS. 14 and 21 and at 52 in FIG. 2. As shown in FIGS. 14 and 20, progress of completion of plural learning modules 56 (e.g., all available learning modules, or a selected or prescribed subset of learning modules 56) can be provided. FIGS. 15, 16 and 17 illustrate, respectively, a selected lesson, progress toward lesson completion, and brief assessment of content understanding at the end of the lesson. If the user enters “no” as an answer, the platform 40 can be configured to automatically recommend another lesson 56 or topic 58 and send a notification to the patient's HCP. FIG. 18 illustrates an example web page providing more detailed assessment of the user's knowledge of one or more lessons 56 and topics 58 (e.g., questions regarding content and grading of answers entered by the user versus merely asking the user their opinion of their level of understanding of lesson content). FIG. 19 illustrates a web page showing the user's profile, notifications and awards earned upon completion of various learning modules, or from attaining desirable glycemic control numbers. FIGS. 20 and 21 illustrate examples, respectively, of the home page (48 in FIG. 2) and “All Courses” page (52 in FIG. 2) in a laptop or tablet form factor rather than a mobile app form factor as shown in FIGS. 13 and 14, and also illustrate a progress bar showing status of completion of lesson modules 56.

### Learning Module Access Codes

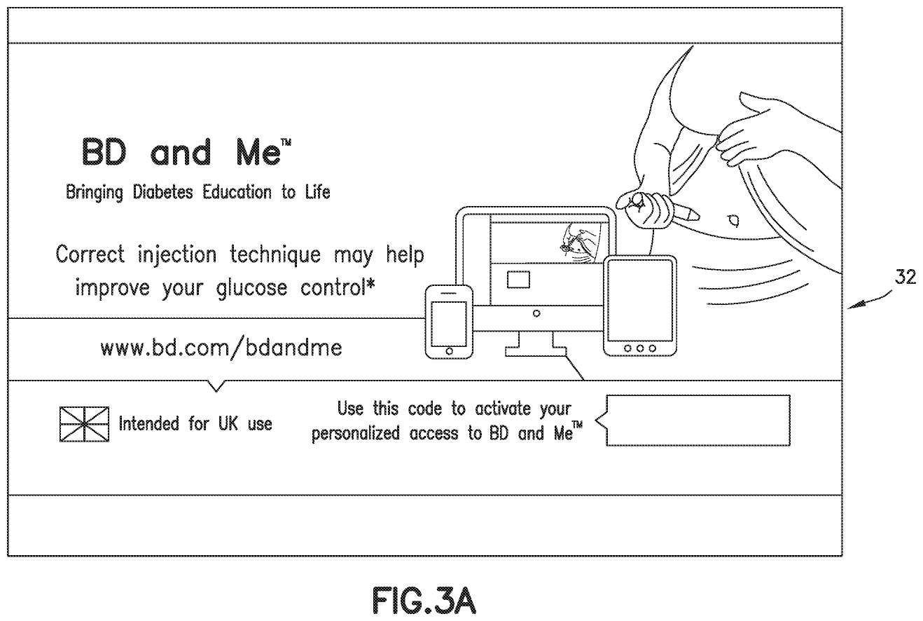

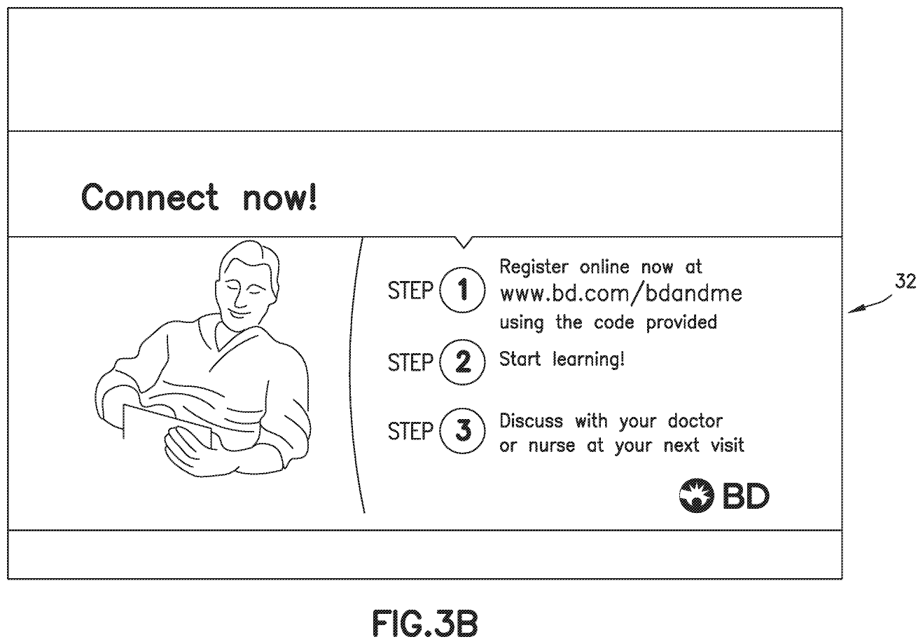

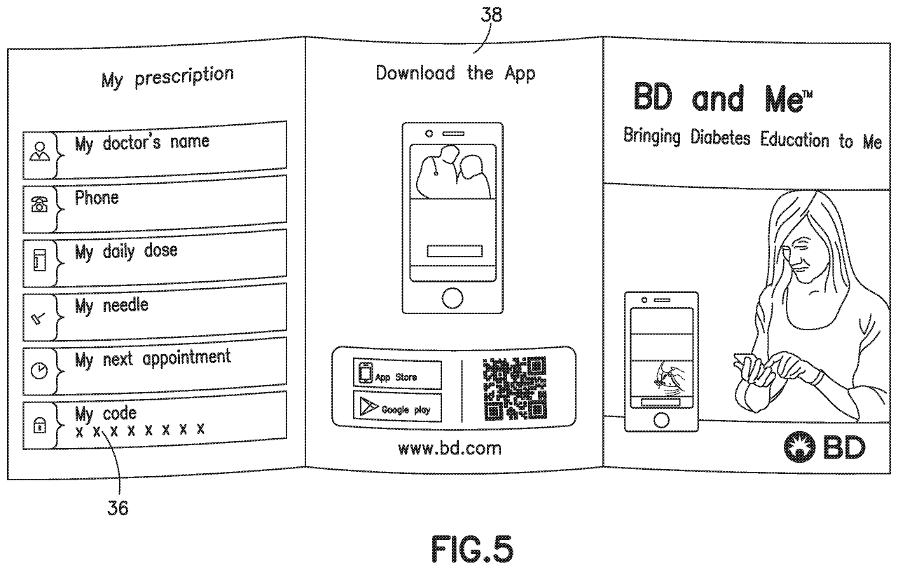

The code can be, for example, an alphanumeric code or employ another nomenclature. The code source 18 that generates the codes, in turn, provides them to product manufacturers and/or suppliers for printing on the products themselves or on a leaflet or other printed item placed within the product packaging. Examples are provided in FIGS. 3A, 3B, 4 and 5. FIGS. 3A and 3B are the respective front and back sides of a printed insert 32 provided for a product's packaging (e.g., inside a box of pen needles). The insert 32 has a code, a uniform resource locator (URL) for the platform 40 and instructions for registering with the platform and entering the code. FIG. 4 depicts a code 36 provided on the exterior of a product package 34 that, in turn, is entered into an area on a web page or screen 30 on a user device 14 for transmission to the platform 40. FIG. 5 is a printed leaflet 38 given to a patient by an HCP. The leaflet can have, for example, sections for an HCP to fill in a code 36 for use by the patient to register with the platform 40. In this instance, the code is not necessarily associated with a particular medical product.

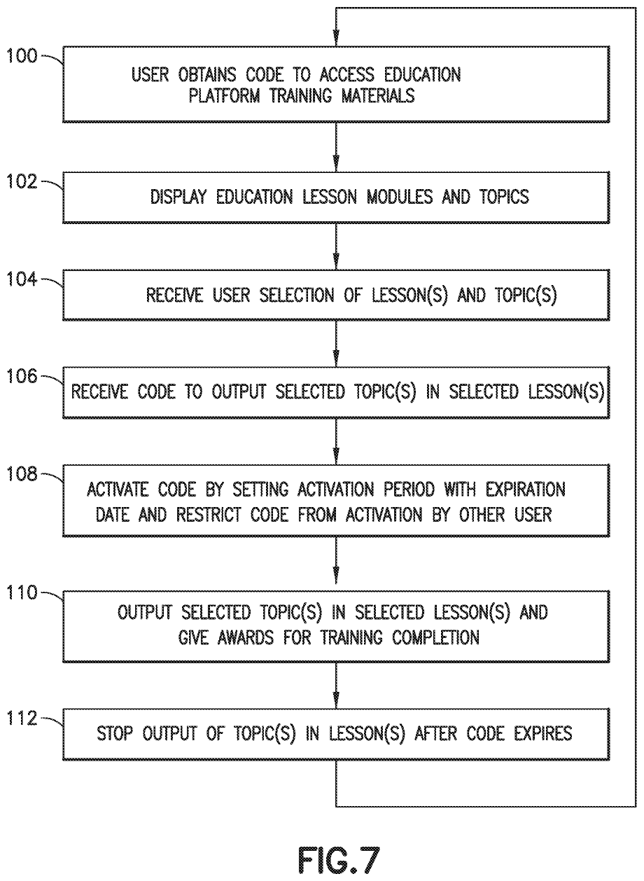

Each code is unique, and the platform 40 with database and backend infrastructure at the server 12 (e.g., code database 168 and user registration and code management module 178 in FIG. 23) is configured to not allow a code to be registered to more than one user at a time. For example, when a code from an HCP or product packaging is entered into a platform 40 web page by a patient, the platform activates that code for a selected period of time (e.g., three months), after which the code automatically expires. In other words, the user has access to the learning modules of the platform 40 for repeated viewing as many times as desired within the three month period. After the code expires, the user will not have access to the learning modules in the platform 40 again until a different code is received (e.g., via a product box with code, or given by an HCP) and activated by entering the code into a platform web page, as described below in connection with FIG. 7).

### Digital Education Platform Website

With continued reference to FIG. 2, the platform web pages can include, but are not limited to, a landing page 42 that can be a scrolling page with both a patient registration portion 44 and a HCP registration portion 46, or can direct these users to respective registration pages 44 and 46. Registration by either a patient or an HCP requires entry of some information such as an e-mail address and creation of a password. Patients can also be required to enter a code (e.g., a code 36 received via a product or an HCP). As stated above, payers can also register with the platform 40 and can have a separate registration/log-in page or portion of a page.

For example, upon registration, the following fields can be required for Patient Data stored and used via the platform 40:

Your country * (drop down list)

Username *

E-mail address *

Password *

Pen Needle box or other product box number *

How did you hear about the platform 40? (drop down list)

I accept the terms and conditions and privacy statement and I′m over 18 *

where * denotes required fields.

After registration, the user receives an email with a link to activate their account.

Upon registration, the following fields can be required for HCP Data stored ans used via the platform 40:

Title *

First name *

Last name *

E-mail address*

Password *

Your job title * (drop down list)

Place of work * (drop down list)

Professional address

Would you like to receive further information on selected products and services? (Opt in)

I accept the terms and conditions and privacy statement *

where * denotes required fields.

After registration, the user receives an e-mail with a link to activate their account.

With regard to data, data security and backend management for the platform 40, the registration data can be encrypted (e.g., using AES 256-bits specifications) after registration, and the encrypted data is stored in the website database associated with the platform 40.

After a user registers with the platform 40, a home page 48 is provided that gives a number of choices such as a main menu 50 indicated in the education content (e.g., learning modules and their respective topics). In an alternative embodiment, the patient code can be entered from a web page or portion of a web page 54 after the main menu 50, instead of having to enter the code at or prior to the home page 48. This way, users have access to other features of the platform besides the educational content such as a listing of the available educational content 52, a workbook page 62, and their profile 64, as well as notifications 66, past workbook entries 68, and awards 70) even if they only have an expired code.

### EXAMPLE LEARNING MODULES

As shown in FIG. 2, the educational content can consist of a number of learning modules or lessons 56 and their respective topics indicated generally at 58. The following is an example listing of diabetes management related topics, focusing on injection technique education. It is to be understood that more or less learning modules or lessons 56 can be provided with more or less topics 58. Also, the learning modules and lessons 58 can be adapted to other types of medical condition management, and to other altogether different content (e.g., learning modules related to a non-medical industry and associated with a particular type of equipment or non-medical product for which training is important for proper and safe use).

Platform 40 learning modules or lessons 56 and topics 58 can be as follows:

### Lesson 1=Preparing to Inject with the Pen

Topic 1: Getting started
About to inject message
Prepare your pen and pen needle
Clean site (wash hands)
Summary

Topic 2: How to use an insulin pen and BD pen needle
New needle
Remove cap
Re-mixing
Fix Pen Needle on the axis of the pen
   Test dose
   Setting the dose
   Injecting
   Giving the insulin
   Needle removal and safe disposal
   Summary

Topic 3: New needle everytime can help keep you healthy
Reusing pen needles can lead to tissue damage
Damage to the needle
Needle blockage
Increased pain or discomfort

### Lesson 2=Where does Insulin need to go

Topic 4: How insulin works

Topic 5: what is under the skin
Skin thickness
Fat
Muscle layer
Needle length

Topic 6: Where on the body to inject
Injection sites
Rotation

### Lesson 3=Choosing the Right Needle Length for you and when and how to Lift a Skin Fold

Topic 7: using the right needle length improve your comfort as well as helping to get the insulin in the right place

Topic 8: Accidental injection into muscle may cause hypos

### Lesson 4=Lumps and Bumps (Lipohypertrophy) can Lead to Blood Glucose Ups and Downs.

Topic 9: What is a lipohypertrophy and what effect does it have.

Topic 10: How to find lipos on your body

Topic 11: Best way to protect yourself from the effects of lipohypertrophy.

Topic 12: How can you prevent lipos developing

Topic 13: How to lift a skin fold and minimise accidental injection into muscle

### Lesson 5=Caring for your Injection Sites

Topic 14: Keep injection sites health, they have to last you a lifetime
Repeated injections lead to Lipos

Topic 15: 6 steps to healthy injection sites

Topic 16: New needle everytime can help keep you beaky. Reusing pen needlescan lead to tissue damage
Damage to the needle
Needle blockage
Increased pain or discomfort

### Lesson 6=How Different Insulins are Absorbed by your Body

Topic 17: What are the main types of insulin that you may use
Repeated injections lead to Lipos

Topic 18: Human insulin time and sites rules

Topic 19: Pre-mix insulin time and sites rules

Topic 20: analog insulin time and sites rules

Topic 21 (optional): Byetta rules (glp1 instead of Byetta)

### Lesson 7=Injection Technique: Dealing with the Unexpected

Topic 22: Bleeding and bruising at your injection sites

Topic 23: Insulin on the skin surface or dripping from the needle after injection

Topic 24: Pain or discomfort

Topic 25: Unexplained hypos and blood glucose ups and down

### Lesson 8=How to Store your Insulin and Equipment and Dispose of it Safely

Topic 26: How to store your insulin

Topic 27: How to safely dispose of your needles and other items

### Learning Module Access

Access to the learning modules 56 and corresponding topics 58 is related to registration with a code (e.g., a code 36 that is included in a prescribed product such as pen needle boxes or given by an HCP). There are different ways the patient can access the platform 40 such as autonomously, or with HCP guidance.

As stated above, user devices 14, 20 can be PCs, tablets and mobile phones that can access the platform 40 via internet and/or cellular network(s) 16. As shown in FIG. 22, the user device 14, 20 has a processor 150 and memory 156 standard for a PC, tablet or mobile phone, as well as at least one communication interface 152 to the one or more networks 16. The memory 156 can store, for example a web browser and/or an app 158 (e.g., a personalized learning portal app) to connect the user to the platform 40. A display 30 is provided for displaying GUI screens in accordance with the app 158 associated with the platform 40), and a user input interface 154 such as a keypad.

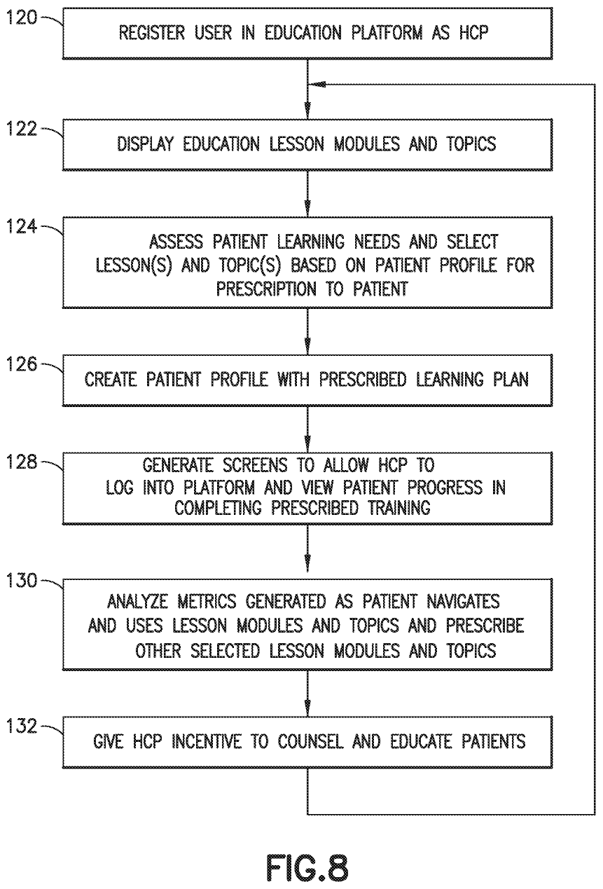

As stated above, the platform 40 is web-based and can be hosted on a server 12. The platform 40 comprises one or more databases for storing the learning modules 56, the codes 36 and user information (e.g., registered user information and user profiles). With reference to FIG. 23, an example platform 40 is depicted with a learning modules database 162, a code database 168 and a user database 170. It is to be understood, however, that these databases are not necessarily discrete memory devices. The platform 40 also comprises a user interface module 164 having a graphical user interface (GUI) module 166 (e.g., for generating the web pages such as those illustrated in FIGS. 9-21) for controlling exchanges between the user devices 14, 20 and the platform 40. The databases 162, 168, 170 and the user interface module 164 are connected to an education platform engine 160 configured to perform platform 40 operations such as the operations described below in connection with FIGS. 7 and 8.

FIG. 7 illustrates example operations of the web-based platform 40 registering a user (e.g., a patient or caregiver) wishing to access the platform 40 via their device 14, and providing access to learning modules 56 via their device 14, and managing activation and deactivation of code(s) 36 entered by the user. With reference to FIG. 7, a patient can access the learning modules 56 of the platform 40 autonomously (i.e., without HCP guidance) by obtaining a box of pen needles or other product via the usual channel (e.g., a pharmacy or drugstore) (block 100), and registering on the platform 40 from their mobile phone, laptop or tablet device 14 by using the access code 36 found, for example, in the box 34 of pen needles. For example, the patient can access the platform 40 using their device 14's web browser and a link to a web-site for the web-based education platform 40. Alternatively, the platform 40 can be accessed via a personalized learning portal app (e.g., app 158 in FIG. 22) downloaded and installed on the user device 14. The platform 40 is provided with a user interface 164 (FIG. 23) having, for example, a graphical user interface (GUI) module 166, to generate screens (e.g., FIGS. 9-21) for display on the user devices 14, 20. For example, the platform 40 is configured to generate a landing page (e.g., FIG. 10), for example. The landing page can optionally provide a listing of available learning modules 56 (block 102), or simply direct the user to first complete a registration process (e.g., initiated via a patient registration page as illustrated in FIG. 11) and not display available learning modules 56 until registration is complete and, optionally, not until a code 36 is entered and activated by the platform 40 as described below.

As stated above in connection with FIG. 2, the platform 40 can allow users to see the learning modules and make selections (blocks 102 and 104) via the educational content menu web page 52, but not have access to the selected content until a valid code 36 is received (block 106). The platform 40 activates the entered code 36 for a selected time period (block 108), and the code 36 will no longer be valid for use by another user. The user can view the selected topics as many times as desired within the selected time period. The education platform engine 160 is provided with a user registration and code management module 178 that works in conjunction with the codes database 168 and with registered users information 180 stored in the user database 170 to control which codes 36 are used by which patients, and when the codes 36 are activated and deactivated and reassigned to other users or retired.

With continued reference to FIG. 7, the platform 40 can be configured to give awards (e.g., badges and opportunities to share awards on social media, discounts on products) to the user for the number of lessons and topics 56, 58 completed and/or for improved patient outcomes (e.g., better glycemic control data such as lowered A1C level based on data provided in the patient profile by the patient or by their HCP) (block 110). In accordance with another aspect of this illustrative embodiment of the present invention, the platform 40 can be used by payers to reward patients for completing training for voluntarily reviewing some or all of the learning modules 56, or as prescribed by their HCP as explained below in connection with FIG. 8. For example, payers can get access to patient profile information indicating learning module completion statistics generated by the platform 40, and/or glycemic control information for that HCP's patients enrolled in the platform 40. A patient incentive can be, for example, the payer reducing the patient's premiums if they reach a selected milestone with their glycemic data or if they simply complete a certain number of the lessons modules 56 within a selected period of time. Alternatively, the payer or a product supplier can provide the patient with coupons for a reduction in the cost of a selected product. The platform 40 is configured to terminate access to the learning modules and topics upon expiry of the code and until another code is obtained by the patient (e.g., from a product or from the patient's HCP) and activated by the platform 40 (block 112).

The patient works through each learning module or lesson 56 of his choice to meet his own interests or perceived learning needs. The patient meets with his HCP at routine consultations and may or may not discuss any of the education platform 40 and his progress with him. For example, the platform 40 can allow the patient to register his HCP through the patient's registration page and profile. The HCP, if registered by the patient, may choose to support the patient by accessing the platform 40, making assessments of that patient's learning, and working with the patient to support recommended further learning with HCP selections of recommended lessons 56 and topics 58.

With reference to FIG. 8 and in accordance with another embodiment of the present invention, the HCP 20 provides guidance to the patient 14 regarding tailored and individualized training using HCP-selected (e.g., prescribed) ones of the lesson modules 56 and topics 58. FIG. 8 illustrates example operations of the web-based platform 40 registering a HCP or other stakeholder (i.e., besides the patient or caregiver) wishing to access the platform 40 via their device 20, providing HCP access to patient profiles when patient consent is given, and facilitating prescription of learning modules 56 to patients. For example, the HCP can access the platform 40 using their device 20's web browser and the link to the web-site for the web-based education platform 40. Alternatively, the platform 40 can be accessed via an education platform app (e.g., app 158 in FIG. 22) downloaded and installed on the user device 20. The platform 40 is configured to generate a landing page (e.g., FIG. 10), for example. The landing page can optionally provide a listing of available learning modules 56 (block 102), or simply direct the HCP to first complete an HCP registration process (e.g., initiated via a HCP registration page as illustrated in FIG. 12) and not display available learning modules 56 until HCP registration is complete. The platform 40 registers the HCP or other stakeholder as an HCP (i.e., as opposed to a patient) (block 120). It is understood that other stakeholders than HCPs can have a different registration page, and that the platform 40 is configured to provide different stakeholders with different privileges. For example, a registered HCP can have access to a patient profile (e.g., if the patient has consented) and to the learning modules 56, whereas a payer or other stakeholder can be registered as a payer who only has access to more generalized metrics available from the platform 40 such as summaries of patient population outcomes (e.g., A1C level reductions achieved by selected populations of patients, which can be organized according to a selected HCP treating those patients), or learning module 56 completion metrics for a selected patient population).

With continued reference to FIG. 8, once registration is completed, the platform 40 is configured to provide the HCP 20 with access to the available lesson modules 56 and topics 58 available to patients (block 122). During a patient consultation, the HCP 20 assesses that patient's learning needs for insulin injection technique and other related diabetes management topics such as preventing lipohypertrophy and recommends one or more lessons 56 and topics 58 (block 124). Alternatively, the platform 40 is configured with an education platform engine 160 (FIG. 23) having a learning module assignment module 172 that is programmed to automatically recommend learning modules 56 for a selected patient based on selected ranges of metrics available from patient profile information (e.g., age, education level, disease management metrics such as glucose readings and/or injection history, progress of medical condition, and so on) and on tracking statistics generated at the platform 40 based on patients' completion of learning modules 56. As stated above, the HCP 20 can give the patient 14 an access code 36 to the prescribed learning material or content available via the platform 40. For example, the HCP 20 can give the patient 14 a leaflet 38 with instructions for the platform 40's use (e.g., URL) and an individualized tailored learning plan to be completed (e.g., a prescribed subset of learning modules 56 and optionally a prescribed subset of topics 58). The platform 40 is configured to receive an individualized learning plan from an HCP or automatically generate a plan, and to store the plan in the patient's profile or in a temporary profile (block 126). For example, an individualized tailored learning plan can be preset for the patient 14 (e.g., based on a patient identifier such as a patient number and/or their e-mail address) and waiting for that patient in a partial patient profile (e.g., that is stored with user profiles 182 in the user database 170 of the at the platform 40) that is completed and activated once the patient registers with the platform 40.

Once the patient 14 registers on the platform from her mobile phone, laptop or tablet or PC 14 and enters the access code 36, the patient is given the goal of completing the planned learning in the time frame recommended by the HCP 20. The platform 40 is provided with a user interface 164 (FIG. 23) having, for example, GUI module 166, to generate screens (e.g., FIGS. 9-21) for display on the user devices 14, 20. For example, the platform 40 can generate screens to allow the HCP to login to the platform 40 and review their patient's progress in completing the prescribed training (block 128). In addition, the HCP 20 and patient 14 can review together the learning completed thus far via the platform 40 in a consultation. Either way, the HCP 20 can make a further assessment of the patient's learning needs and recommend further lessons 56 and topics 58 as required (block 130). As stated above, the platform 40 is configured with an engine 160 (FIG. 23) having a learning module assignment module 172 that is programmed to automatically recommend additional learning modules 56 for a patient once prescribed modules have been completed and based on various metrics.

In accordance with another aspect of this illustrative embodiment of the present invention, the platform 40 can be used by payers to reward HCPs 20 for counseling and educating patients. For example, the platform engine 160 comprises an incentive determination module 174 configured to analyze patient profile information (e.g., outcomes and status of medical condition management based on selected parameters being within designated ranges) and/or progress of completion of learning modules by the patients of a selected HCP and determine a reward for the patient and/or HCP when designated metrics are achieved (block 132). In addition, payers can get access to generalized patient profile information indicating learning module completion statistics generated by the platform 40 (e.g., by a user navigation and progress tracking module 176 in the engine 160), and/or glycemic control information for that HCP's patients enrolled in the platform 40 (e.g., stored in user profiles 182 in the user database 170). An incentive can be, for example, authorization for the HCP to use CPC codes that provide a greater amount of compensation for patient education and counseling visits.

In accordance with another embodiment of the present invention, more interaction between the HCP 20 and the patient 14 is supported by the platform 40. For example, the HCP 20 assesses patient's learning needs and explains the benefits and processes of the platform 40 to the patient 14. The patient 14 is then invited by the HCP to complete a tailored learning plan via the platform 40. The HCP asks the patient for an e-mail address with which the HCP creates a new patient process with specific learning lessons and profiles within the platform 40. The platform 40, in turn, sends and an e-mail invite with a link for the platform 40 to the patient, who can then click on the link and access any of the lessons or topics in his prescribed tailored learning plan at a time convenient to him. The patient is merely requested by the platform 40 to register with the code 36 found in his box of pen needles or other product or otherwise given to him by his HCP to gain full access to the platform 40. In this illustrative embodiment, the HCP is directly linked to patient account and has visibility on the patient's profile. The HCP can follow patient activity and progress (e.g., connections to the prescribed learning modules and topics tracked via the user navigation and progress tracking module 176 in the engine 160, and assessments generated via the platform 40, the completion of which by the patient indicates to what extent the patient understands a particular topic), and is able to provide special support to the patient in clinic visits when needed or to prescribe other learning modules 56 and topics 58. The patient works through a prescribed tailored learning program as planned by his HCP, receiving push messages as requested by HCP or automatically sent via the platform 40. During a routine consultation, the patient and HCP can review the learning completed thus far. The HCP can, in turn, choose to support the patient by making a further assessment of the patient's learning needs and working with that patient to support recommended further learning. As stated above, the platform engine 160 is configured with a learning module assignment module 172 that is programmed to automatically recommend learning modules 56 for a selected patient based on selected ranges of metrics available from patient profile information (e.g., age, education level, disease management metrics such as glucose readings and/or injection history, progress of medical condition, and so on) and on tracking statistics generated at the platform 40 based on patients' completion of learning modules 56 and analyzed by the user navigation and progress tracking module 176.

In accordance with an illustrative aspect of the platform 40, the platform 40 can generate one or more dashboards for the HCP 20 via the engine 160 and GUI module 166 of the user interface module 164. For example, the data collected by the platform 40 from the HCP 20 and his registered patients 14 can be used to create a single screen graphical user interface (GUI) dashboard that shows the HCP's patients that have registered to the platform 40, which lessons and topics have been accessed, and numbers of times lessons and topics have been viewed and percentage of completion, and so on.

As stated above, in accordance with illustrative embodiments of the present invention, the platform 40 is configured as a smart platform 40 having an engine 160 with algorithms to make automated assessment of the patient's learning needs based on patient profile information (e.g., glycemic control information and prescribed insulin regimen) and provide a prescribed tailored learning plan with selected ones of the lessons 56 and topics 58. For example, selected parameters or ranges relating to different glycemic control criteria, different insulin types, different injection criteria, lipohypertrophy location(s) and history, and the like are used with corresponding information obtained from the patient profile (e.g., stored in user profiles 182 in the user database 170) to generate a list of recommended lessons 56 and topics 58. Certain lessons 56 with certain topics 58 are prescribed, for example, if the patient has never self-injected medication before (e.g., Lesson 1 described above), while a patient with a history of injecting for over a year may need training on how to find lumps and bumps at injection site indicative of lipohypertrophy (e.g., Lesson 2, Topic 6 on injection site rotation, and Lesson 4, described above).

As stated above, in accordance with another aspect of the present invention, the platform 40 or other related system generates content access metrics as the patient navigates and uses lesson modules 56 and topics 58 (e.g., via the user navigation and progress tracking module 176). For example, the platform 40 can generate individual user analytics which can include, but are not limited to, statistics relating to log-in frequency (e.g., the number of times a patient has logged into the platform 40), lessons 56 and topics 58 accessed and completed, duration and timing of access to the platform 40 in general or to a particular lesson 56 and topic 58, dwell times and viewing activity, mouse clicks and movement, and so on. This analytical data relating to user access and usage of the platform 40 enables comparisons of education platform usage and use of analytics and clinical and behavioral outcomes to determine treatment or training compliance or training comprehension issues.

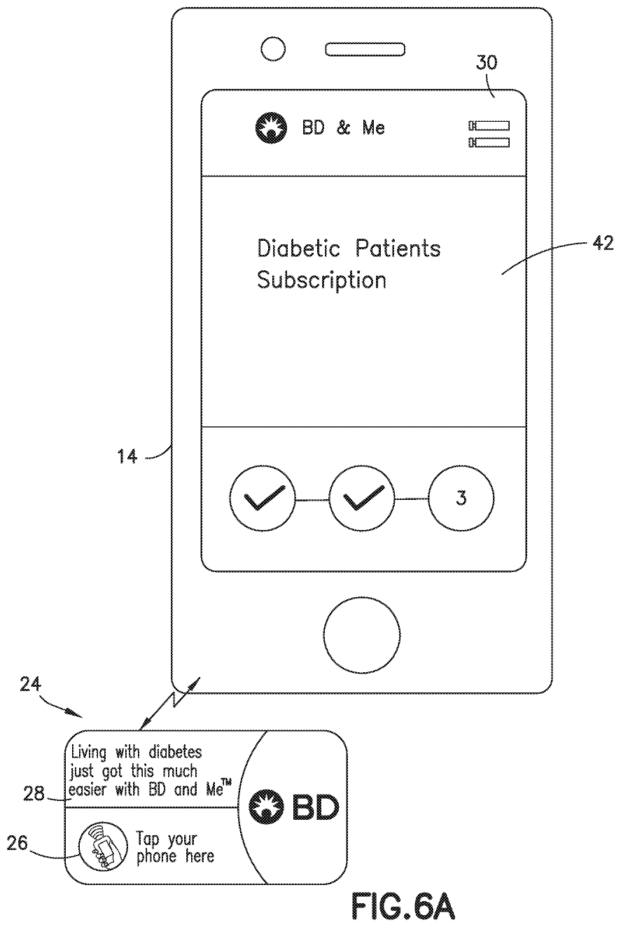

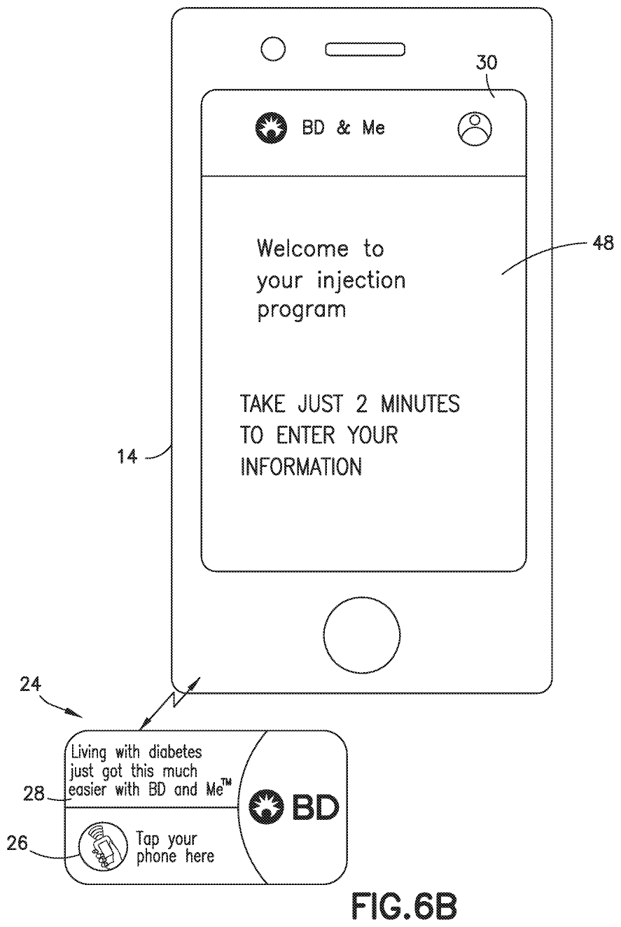

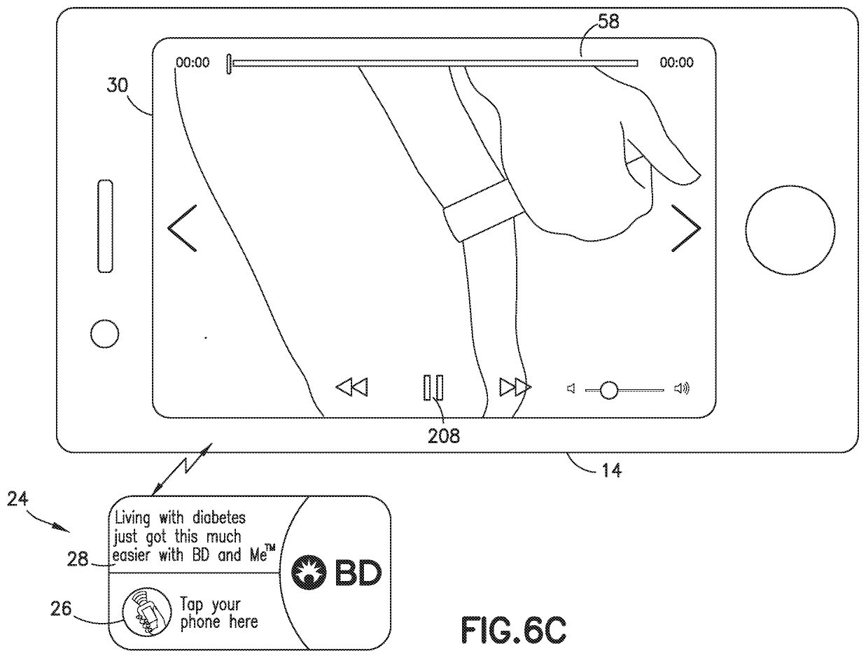

With reference to FIGS. 6A, 6B and 6C and in accordance with another illustrative embodiment of the present invention, the product, or a nearby shelf on which similar products are displayed, can receive a label or other device 24 configured as described in WO 2017/083262. The label 24 is designed to generate viewable content (e.g., static content such as graphic display, or dynamic content such video), and is a self-powered device at a point of interaction with one or more users that employs ambient radio frequency energy harvesting to charge a renewable, rechargeable energy storage element.

For example, as shown in FIGS. 6A, 6B and 6C, the device 24 can display a static image. The display area shown in FIGS. 6A, 6B and 6C is essentially the total front surface area of the device 24. The device 24, however, can be configured to have a smaller display area than its viewable front surface area. The device 24 can be provided with a user input device area 26 on the viewable front surface area that can be a part of, or separate from, the display 28 provided on the device 24. For example, the display 28 of device 24 can be a LCD display capable of touchscreen input from a displayed prompt (e.g., “Tap your phone here” and corresponding icon 26). Since the device 24 operates in conjunction with a smart phone or other portable device 14 that presumably has a color LCD or LED display and touchscreen inputs or other device inputs for video viewing control operations (e.g., pause, resume, play, rewind or skip backward, and fast forward and skip forward), the display 28 on the device 24 need only output static images and can therefore be a relatively inexpensive display if desired such as an Electronic Paper Display (EPD) using E-ink technology.

With continued reference to FIGS. 6A, 6B and 6C, the device 24 can be programmed to communicate with the smart phone or other portable device 14 using a wireless communication protocol such as, for example, RFID or other near field communication (NFC) protocol or Bluetooth™ to pair or otherwise recognize the smart phone or other portable device 14 when it is brought into close proximity to the device 24 (e.g., tapping the user input with a smart phone 14). In response to the device 24 acknowledging or pairing with the smart phone 14, a processor in the device 24 is configured to stream selected dynamic content stored at the device 24 to the smart phone or other portable device 14 for playback on its display 30. This is advantageous since many existing smart phones and devices are not NFC-enabled. Such devices have typically been required to have an NFC application installed on them to first energize and communicate with an RFID tag, for example, to receive merely a website link. These devices would then have to navigate to a browser to use the link received from the RFID tag for playback. Proposed NFC-enabled devices, while more automated and requiring less user configuration, will still similarly need to download desired content from the internet to playback that content. The device 24, by contrast, advantageously directly sends desired stored content such as a video segment, or alternatively a series of static images, to the smart phone or device 14 for immediate playback on its display 30 without having to look up content on the internet. Accordingly, the device 24 can send a brief tutorial or promotion regarding the platform 40 and its benefits to patients if they register. Alternatively, the device 24 can simply send the URL for the platform landing page 42 which is then displayed on the device 14 as shown in FIG. 6A. Either way, the device 24 can facilitate navigation to and display of a landing page 42 (FIG. 6A) or a home page 48 (FIG. 6B) on a user device 14.

In addition, the device 24 can automatically send a code 36 stored thereon to the user device 14, or display the code on its display 28, for entry into the registration page or other page of the platform 40 accessed via the mobile phone 14. With continued reference to FIG. 6C, as a result of the label 24 giving a user automated access to the platform 40 via a user device 14 at a point of interaction, the user is able to discern the added education benefits of a particular product and its related platform. In addition, the user device 12 is able to playback the selected lessons 56 and topics 58 on its display 30, and can be required to enter the code 36 from the label or device 24 in order to do so.

### Learning Module Analytics and Training Incentives

As stated above, patients can earn badges as they complete lessons 56 and topics 58 to encourage users in their progression and training. To monitor website metrics, the platform 40 can employ a “Google Analytics” service provided by Google or similar tool as at least part of the user navigation and progress tracking module 176. Detailed Google Analytics features can be accessed at the following address: http://www.google.com/analytics/. The platform 40 can also employ “Hotjar” or similar tool dedicated to user experience analysis. Hotjar can, for example, provide the following information to help improve website ergonomy and use: User journeys on the site and registration forms, and a “Heat Map.”

Patient or individual user analytics can include, but are not limited to, log on frequency, courses accessed and completed, lessons accessed and completed, duration and timing of access, dwell times and viewing activity, mouse clicks and movement. Such data enables comparisons of education platform usage analytics with clinical and behavioral outcomes to determine compliance or comprehension issues. Accordingly, HCPs have a basis by which to prescribe other modules or in person training if repeated viewings are logged and a patient's glycemic control data is unsatisfactory.

The digital education platform 40 of the present invention is an improvement to diabetes management and education on a patient's own schedule. All platform 40 content is available on demand, that is, it is ready when the patient is ready. A patient can now engage with information from a global expert who created the platform content 56, 58 and is at the leading edge of medical condition management. Most sessions are interactive and offer a live Q&A. Patients can transform their delivery routines with information and insights that deliver science-driven safety and efficiency for healthy patient outcomes. The GUI described herein and the patient experience provided by the platform 40 allows the user to benefit from self-directed and engaging tutorials, and can provide a customized scrolling path through content for that patient such that the user can walk through educative imperatives at different stages and at their own pace.

In addition, the digital education platform 40 serves multiple stakeholders and can be provided for medical education such as diabetes management including injection regimens and procedures to minimize pain, increase injection efficacy and reduce lipohypertrophy and increase regimen compliance.

The components of the illustrative devices, systems and methods employed in accordance with the illustrated embodiments of the present invention can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the present invention can be easily construed as within the scope of the invention by programmers skilled in the art to which the present invention pertains. Method steps associated with the illustrative embodiments of the present invention can be performed by one or more programmable processors executing a computer program, code or instructions to perform functions (e.g., by operating on input data and/or generating an output). Method steps can also be performed by, and apparatus of the invention can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof

Those of skill in the art further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present invention. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

The above-presented description and figures are intended by way of example only and are not intended to limit the present invention in any way except as set forth in the following claims. It is particularly noted that persons skilled in the art can readily combine the various technical aspects of the various elements of the various illustrative embodiments that have been described above in numerous other ways, all of which are considered to be within the scope of the invention.

1. A method of accessing tailored information from a web-based education platform comprising:
storing a plurality of indexed learning modules in a memory storage device;
storing a plurality of codes in a memory storage device;
registering a user with the platform;
receiving a code entered by the user into a user portal to the platform;
determining if the entered code is one of the plurality of codes;
permitting the user access into the platform and to the indexed education learning modules when the entered code is determined to be from among the plurality of codes; and
generating an output of a selected subset of the indexed education learning modules based on one of user selection of the subset of the indexed education learning modules, and selection of the subset by a provider who prescribes training to the user.

2. The method of claim 1, wherein the learning modules comprise any of video, multimedia, and print materials.

3. The method of claim 1, wherein the user is a patient and the provider is a healthcare provider (HCP), the learning modules are divided by different lessons relating to different aspects or stages of medical injection, and each lesson has plural topics, and the HCP selects the subset based on patient data related to medical condition management.

4. The method of claim 3, further comprising
generating user metrics relating to a plurality of criteria selected from the group consisting of login frequency, lessons accessed and completed, topics accessed and completed, duration and timing of lesson or topic accesses, dwell times and mouse activity during lesson or topic accesses;
wherein the HCP prescribes different ones of the lessons and topics based on the user metrics.

5. The method of claim 3, wherein receiving a code comprises the user getting a code from an injection product or from a healthcare provider (HCP) and entering it into a user interface screen of the platform.

6. The method of claim 1, wherein registering comprises setting a user profile.

7. The method of claim 6, wherein registering comprises a user granting an HCP access to the user profile.

8. The method of claim 1, further comprising:
activating a code after it is entered into the platform for a selected period of time;
providing the user with unlimited access to the indexed education learning modules during the selected period of time;
expiring the code after the selected period of time has elapsed; and
denying the user access to the indexed education learning modules after the code has expired.

9. The method of claim 8, further comprising
obtaining a second code; and
entering the second code into the platform portal to gain permission to access to the indexed education learning modules again for a selected period of time until the second code expires.

10. The method of claim 9, wherein the education learning modules are divided by different lessons relating to different aspects or stages of medical injection, and each lesson has plural topics, and further comprising
generating user metrics relating to a plurality of criteria selected from the group consisting of login frequency, lessons accessed and completed, topics accessed and completed, duration and timing of lesson or topic accesses, dwell times and mouse activity during lesson or topic accesses; and
the HCP reviewing the user metrics generated during the respective selected time periods related to the code and second code and prescribing different ones of the lessons and topics based on the user metrics.

11. The method of claim 10, wherein the HCP prescribes different ones of the lessons and topics based on patient data related to medical condition management.

12. The method of claim 1, further comprising
generating user metrics relating to a plurality of criteria selected from the group consisting of login frequency, lessons accessed and completed, topics accessed and completed, duration and timing of lesson or topic accesses, dwell times and mouse activity during lesson or topic accesses;
storing patient data related to medical condition management;
providing payers access to the user metrics and patient data; and
rewarding at least one of a patient and healthcare provider with incentives to encourage the patient consumption of the indexed education learning modules.

13. The method of claim 1, further comprising
operating an energy harvesting display device to display content regarding the platform;
wirelessly coupling the display device with a mobile phone and transmitting stored content in the display device to the mobile phone; and
playing the stored content from the display device on the mobile phone.

14. The method of claim 13, further comprising
providing the mobile phone with a graphical user interface (GUI) to the platform;
generating a GUI screen with a field in which to enter the code; and
playing back selected ones of the subset of the indexed education learning modules via the mobile phone only after the code is entered.

15. The method of claim 14, further comprising
placing the energy harvesting display device on a product, wherein the product has a unique code stored in the energy harvesting display device; and
performing at least one of displaying the code for the user to manually enter into the field, and automatically filling the field with the code by the energy harvesting display device.

16. A method of providing individualized information to a user from a web-based education platform comprising:
storing a plurality of indexed learning modules in a database, the learning modules comprising respective educational content;
storing a plurality of codes in a database;
restricting access to the learning modules until a code from among the plurality of codes has been received and activated;
receiving a first signal indicative of a user registration with the platform and, in response to the first signal, generating a user profile for that user comprising at least user identification and storing the user profile in the database;
receiving a second signal indicative of a code inputted by the user;
in response to the second signal, determining if the code inputted by the user is from among the plurality of codes and is not activated for another user;
when the code inputted by the user is determined to be from among the plurality of codes and not activated for another user, generating and storing information in the database indicative of association of the code with the user, and configuring the database to prevent the association of that code with a different user;
activating the code for that user for a selected time period;
transmitting a third signal indicative of a selected subset of the indexed learning modules for display to the user on a user device, the selected subset of the indexed learning modules based on one of user selection of the subset of the indexed learning modules and selection of the subset by a provider who prescribes training to the user;
receiving a fourth signal indicative of at least one of the selected subset of the indexed learning modules being identified by the user for display on the user device and, in response to the fourth signal, outputting the identified learning module to the user device; and
in response to expiration of the time period, deactivating the code, and configuring the database to prevent the output of any of the indexed learning modules to the user device until another one of the plurality of codes is inputted by the user and determined to not be activated.

17. The method of claim 16, wherein the learning modules are divided by different lessons and each lesson has plural topics, further comprising
the platform obtaining user metrics relating to a plurality of criteria selected from the group consisting of user login frequency to the platform, lessons accessed and completed, topics accessed and completed, duration and timing of lesson or topic accesses, dwell times and mouse activity during lesson or topic accesses; and
receiving a fifth signal indicative of different ones of the lessons and topics prescribed by the provider based on the user metrics.

18. The method of claim 16, wherein the learning modules are divided by different lessons and each lesson has plural topics, further comprising
the platform obtaining user metrics relating to a plurality of criteria selected from the group consisting of user login frequency to the platform, lessons accessed and completed, topics accessed and completed, duration and timing of lesson or topic accesses, dwell times and mouse activity during lesson or topic accesses; and
analyzing the user metrics to prescribe different ones of the lessons and topics to the user based on the user metrics.

19. The method of claim 16, further comprising:
receiving a fifth signal indicative of the user granting a provider access to the user profile;
receiving a sixth signal indicative of a provider registration with the platform and, in response to the seventh signal, providing the provider with access to the user profile in the database;
transmitting an seventh signal to the provider to display on a provider device the plurality of indexed learning modules;
receiving a eighth signal indicative of a provider-selected subset of the plurality of indexed learning modules and storing them as a prescribed subset of the plurality of indexed learning modules in the user profile.

20. The method of claim 19, further comprising:
analyzing at least one of the user profile, and user metrics relating to a plurality of criteria selected from the group consisting of user login frequency to the platform, learning modules accessed and completed by the user, duration and timing of learning module accesses, dwell times and mouse activity during learning module accesses, and generating a prescribed subset of the plurality of indexed learning modules when designated criteria in at least one of the user profile and the user metrics are satisfied.

21. A web-based education platform connected to least one user device via a communication network, the user device having a user output device and a user input interface, the platform comprising:
a memory storage device comprising instructions;
a plurality of indexed learning modules stored in the memory storage device;
a plurality of codes stored in the memory storage device;
a graphical user interface (GUI) module configured to generate screens for display on the user output device;
at least one processor in communication with the memory storage device and the GUI module, wherein the processor executes the instructions to:
restrict access to the learning modules until a code from among the plurality of codes has been received and activated;
receive a first signal indicative of a user registration of the user device with the platform and, in response to the first signal, generate a user profile for that user comprising at least user identification and storing the user profile in the database;
receive a second signal indicative of a code inputted by the user via the user input interface;
in response to the second signal, determine if the code inputted by the user is from among the plurality of codes and is not activated for another user;
when the code inputted by the user is determined to be from among the plurality of codes and not activated for another user, generate and store information in the memory storage device indicative of association of the code with the user, and configure the memory storage device to prevent the association of that code with a different user;
activate the code for that user for a selected time period;
transmit a third signal indicative of a selected subset of the indexed learning modules for display to the user on a user output device, the selected subset of the indexed learning modules based on one of user selection of the subset of the indexed learning modules and selection of the subset by a provider who prescribes training to the user;
receive a fourth signal indicative of at least one of the selected subset of the indexed learning modules being identified by the user for display on the user output device and, in response to the fourth signal, output the identified learning module to the user device; and
in response to expiration of the time period, deactivate the code, and configure the memory storage device to prevent the output of any of the indexed learning modules to the user device until another one of the plurality of codes is inputted by the user and determined to not be activated.

22. The system of claim 21, wherein the learning modules are divided by different lessons, and each lesson has plural topics, and the processor executes the instructions to obtain user metrics relating to a plurality of criteria selected from the group consisting of login frequency of the user device, lessons accessed and completed via the user device, topics accessed and completed via the user device, duration and timing of lesson or topic accesses by the user device, dwell times and user device mouse activity during lesson or topic accesses.

23. The system of claim 21, wherein the processor executes the instructions to store patient data related to medical condition management in the memory storage device and to provide at least one of a healthcare provider device and a medical claims payer device with access to the user metrics and patient data via the platform.

24. The system of claim 23, wherein the medical claims payer device can provide the platform with incentives to users for completing the indexed learning modules and reward criteria for bestowing the incentives to the users, wherein the processor executes the instructions to analyze the user metrics and provide the incentives to the users when the reward criteria are met.

25. The system of claim 22, further comprising a healthcare provider device configured to receive the user metrics with respect to each of a plurality of user devices, to prescribe access to at least a selected one of the indexed learning module for respective ones of the plurality of user devices based on their corresponding user metrics, and to generate and provide prescriptions for the plurality of user devices to the platform, the processor executes the instructions to correlate the prescriptions for the plurality of user devices with registration information for respective user devices to output the prescribed indexed learning modules.

26. The system of claim 21, wherein the learning modules are divided by different lessons relating to different aspects or stages of medical injection selected from the group consisting of preparing injection device for delivery, selecting injection sites, selecting needle length, detecting lipohypertrophy, caring for injection sites, medication types, injection technique, and storage and disposal of medical injection supplies.

27. The system of claim 21, wherein the processor executes the instructions to activate the access code for a selected period of time after it is entered into the platform portal via the user interface, provide the user device with unlimited access to the indexed learning modules during the selected period of time, retire the access code after the selected period of time has elapsed, and deny the user device access to the indexed learning modules after the access code has expired.

28. The system of claim 27, further comprising a second product having a second access code, wherein the processor executes the instructions to receive the second access code entered via the user input interface, and provide the user device with access to the indexed learning modules again for a selected period of time until the platform retires the second access code.
